(19) United States
(12) Patent Application Publication
(10) Pub. No.: US 2024/0181205 A1
(43) Pub. Date: Jun. 6, 2024

(54) CATHETER AND METHOD FOR PRODUCING SUCH A CATHETER

(71) Applicant: B. Braun Melsungen AG, Melsungen (DE)

(72) Inventors: Egbert Dubielzig, Fritzlar (DE); Oliver Kahlen, Gudensberg (DE); André Weiss, Guxhagen (DE)

(21) Appl. No.: 18/524,365

(22) Filed: Nov. 30, 2023

(30) Foreign Application Priority Data
Dec. 1, 2022 (DE) 10 2022 212 931.8

(51) Int. Cl. A61M 25/00

(52) U.S. Cl. CPC A61M 25/0026; A61M 25/0012; A61M 25/0069; A61M 2025/0018; A61M 2207/10

(57) ABSTRACT

A catheter, which can be used in infusion therapy, includes a catheter tube having a main lumen and at least one secondary lumen. The main lumen and the at least one secondary lumen are each elongate between a proximal tube end and a distal tube end. The at least one secondary lumen has a lateral opening arranged between the proximal tube end and the distal tube end in an axial direction of the catheter tube. The lateral opening extends through a tube casing of the catheter tube in a radial direction of the catheter tube. A seal is introduced into the at least one secondary lumen distally behind the lateral opening and at least partially seals the secondary lumen. The seal is formed from a sealing compound cured in the at least one secondary lumen or a thermoplastic elastomer material expanded within the at least one secondary lumen.

### CROSS-REFERENCE TO RELATED APPLICATION(S)

This application claims priority under 35 U.S.C. § 119 to German Application No. 10 2022 212 931.8, filed on Dec. 1, 2022, the content of which is incorporated by reference herein in its entirety.

### FIELD

The disclosure relates to a catheter comprising a catheter tube which has a main lumen and at least one secondary lumen, the main lumen and the at least one secondary lumen each being elongate between a proximal tube end and a distal tube end, and the at least one secondary lumen having a lateral opening which is arranged between the proximal tube end and the distal tube end in the axial direction of the catheter tube and extends through a tube casing of the catheter tube in the radial direction of the catheter tube, and comprising a seal which is introduced into the at least one secondary lumen distally behind the lateral opening and seals said secondary lumen at least partially.

### BACKGROUND

Such a catheter is generally known in the field of medical technology, for example as a multilumen central venous catheter. The known catheter comprises a catheter tube having a main lumen and at least one secondary lumen. The main lumen and the at least one secondary lumen are each elongate between a proximal tube end and a distal tube end of the catheter tube. The at least one secondary lumen has a lateral opening which extends through a tube casing of the catheter tube in the radial direction. In case of the known catheter, a so-called mandrin is introduced into the at least one secondary lumen in the distal direction behind the lateral opening. For example, the mandrin can be in the form of a rod or cord and acts as a seal. The mandrin is manufactured separately from the catheter tube and is pushed into the at least one secondary lumen in the proximal or distal direction in a separate step during the production of the catheter. The mandrin is pushed in manually.

### SUMMARY

It is an object of the present disclosure to provide a catheter of the type mentioned at the beginning that has a simple structure and, at the same time, improved properties compared to the prior art.

This object is achieved by the seal being formed from a sealing compound introduced into the at least one secondary lumen in a viscous state and cured within the at least one secondary lumen. The solution according to the disclosure can dispense with a mandrin and with pushing said mandrin into the at least one secondary lumen during manufacture. This significantly simplifies the structure of the catheter and the production thereof. The introduction of the (initially) viscous sealing compound into the at least one secondary lumen and the curing of the sealing compound introduced into the at least one secondary lumen to form the seal is automatable by comparatively simple means. This is in contrast to pushing in the mandrin, as known from the prior art. The sealing compound and/or the seal formed from same is arranged in the at least one secondary lumen in the radial direction without any gaps, more precisely on the inner wall of said secondary lumen. As a result of curing, the sealing compound can be integrally bonded to the inner wall of the at least one secondary lumen. As a result of the gap-free arrangement, the tube casing, in the region of the seal, is directly force-transmitting supported thereon. In contrast, mandrins known from the prior art necessarily require a certain radial clearance in order to allow push-in into the at least one secondary lumen during manufacture. In addition, production-related dimensional tolerances and thus additional clearance between the mandrin and the at least one secondary lumen, more precisely the inner wall thereof, may be present. Said radial clearance may lead to insufficient support of the tube casing on the mandrin. Under the action of temperature and/or pressure, this may lead to sink marks in the region of the mandrin in the case of catheters known from the prior art. Such sink marks are undesirable for various reasons. Moreover, in the case of catheters known from the prior art having mandrins, a residual volume often forms in the form of an annular space between the mandrin and the inner wall of the at least one secondary lumen. Such a residual volume is likewise undesirable. What may occur within the residual volume when using the catheter is the accumulation of standing liquid and, as a consequence, an accumulation and/or an increased growth of microbes harmful to health. The disclosure eliminates these disadvantages. Preferably, the sealing compound is arranged in the at least one secondary lumen in the distal direction directly behind the opening. In other words, the seal is directly adjacent to the lateral opening in the axial direction. The catheter tube is elongate between its proximal tube end and its distal tube end. Preferably, the catheter tube is flexible. The catheter tube preferably has a cylindrically elongate main shape with a round, in particular circular, or oval cross-section. The main lumen preferably has an inlet opening arranged at the proximal tube end and an outlet opening arranged at the distal tube end and/or at a catheter tip. The main lumen is elongate between its inlet opening and its outlet opening. The at least one secondary lumen preferably has an inlet opening arranged at the proximal tube end. Moreover, the at least one secondary lumen has the lateral opening which extends radially through the tube casing. In one embodiment, the catheter has a catheter tip which is arranged on the distal tube end and which can be, for example, conical or rounded. The catheter tip can be manufactured as a separate component and then joined, preferably integrally joined, to the distal tube end. Alternatively, the catheter tip can be integrally molded on the catheter tube by means of forming of the distal tube end. In one embodiment, the catheter is a multilumen central venous catheter. However, the solution according to the disclosure is not limited to central venous catheters, but is advantageously also applicable to other multilumen catheters. If the catheter has multiple secondary lumens, for example two, three, four, five or more than five secondary lumens, they are each preferably sealed with a seal formed from the sealing compound.

In one embodiment, the sealing compound is and/or contains cyanoacrylate. Cyanoacrylate is understood to mean polymerizable chemical compounds (monomers) that are liquid at room temperature. Such compounds are often used as adhesive and are known under the names instant glue or superglue. In this embodiment, the sealing compound preferably cures under the action of air humidity. This allows particularly simple production of the catheter. In a cured state, the cyanoacrylate has a comparatively high capacity to withstand stresses. This is associated with various advantages.

In one embodiment, the sealing compound is and/or contains silicone. Just like the cyanoacrylate described above, silicone is curable under the action of air humidity alone. Consequently, this embodiment of the disclosure also allows particularly simple production of the catheter. Moreover, silicone is elastic, resilient and/or flexible in the cured state. This ensures that flexible properties of the catheter tube, if any, are not impaired by the seal formed from the sealing compound.

In one embodiment, the sealing compound is and/or contains a crosslinkable polymer, in particular a resin, a monopolymer and/or a prepolymer. In this embodiment, the sealing compound can therefore be referred to as a reactive substance in the broadest sense. The reactive substance can be cured, for example, under the action of temperature, UV light or the like.

The present disclosure further relates to a method for producing a catheter mentioned at the beginning. The method according to the disclosure comprises the steps of: introducing a viscous sealing compound into the at least one secondary lumen, the viscous sealing compound being introduced into the at least one secondary lumen through the lateral opening; curing the viscous sealing compound introduced into the at least one secondary lumen, the seal being formed by the cured sealing compound. With respect to resulting advantages and further features and embodiments of the method, reference is made to the description of the catheter according to the disclosure and its embodiments. What is stated there also applies, mutatis mutandis, to the method according to the disclosure and its embodiments. The same applies vice versa. The introduction of the viscous sealing compound is effected through the lateral opening and in the distal direction, and so the seal is formed in the distal direction behind the lateral opening. The introduction can be achieved by, for example, injecting and/or pressing the viscous sealing compound into the at least one secondary lumen. The introduction can be done manually using a tool suitable for this purpose, semiautomatically or fully automatically. Depending on the composition of the sealing compound used, the curing can be effected, for example, under the action of air humidity, action of heat and/or action of radiation, in particular action of UV light.

The mentioned object of the disclosure is also achieved by the seal being formed from a thermoplastic elastomer material introduced into the at least one secondary lumen and expanded within the at least one secondary lumen. This solution according to the disclosure can also overcome the disadvantages associated with pushing a mandrin into the at least one secondary lumen. To the knowledge of the inventors, expandable thermoplastic elastomers have only been available on the market for a few years. In the field of medical technology, expandable thermoplastic elastomers are not further known to date. Expandable thermoplastic elastomers are also known under the abbreviation EP-TPE. The composition of such expandable thermoplastic elastomers comprises a blowing agent activatable under the input of heat. The activation of the blowing agent brings about an expansion of the thermoplastic elastomer material. When the EP-TPE is expanded, the original (nonexpanded) volume thereof can be increased many times over. Moreover, a porous morphology is formed. In the expanded state, the thermoplastic elastomer material introduced into the at least one secondary lumen rests against the inner wall of the at least one secondary lumen in the radial direction without any gaps. As already explained, solutions known from the prior art using mandrins usually require a certain radial clearance in order to allow push-in into the at least one secondary lumen during manufacture. This radial clearance remains even after the production process. Said clearance results in the disadvantages already explained. These disadvantages are eliminated by the present solution according to the disclosure. If the catheter has multiple secondary lumens, for example two, three, four, five or more than five secondary lumens, they are each preferably sealed with a seal formed from the expanded thermoplastic elastomer material.

The present disclosure further relates to a method for producing a catheter that comprises the steps of: introducing an expandable thermoplastic elastomer material into the at least one secondary lumen, the expandable thermoplastic elastomer material being introduced into the at least one secondary lumen through the lateral opening; expanding the thermoplastic elastomer material introduced into the at least one secondary lumen, the seal being formed by the expanded thermoplastic elastomer material.

The expandable thermoplastic elastomer material is introduced into the at least one secondary lumen in different forms in different embodiments of the method. In one embodiment, particles of the expandable thermoplastic elastomer material are introduced into the at least one secondary lumen. To this end, the particles are filled into the at least one secondary lumen through the lateral opening. In further embodiments, said particles are processed before introduction into the at least one secondary lumen. Depending on the nature of the processing of the particles, different moldings, semifinished products or the like can be produced for introduction into the at least one secondary lumen. In one embodiment, the particles are processed into an expandable shaped cord, filling or the like. In a further embodiment, the particles are filled into an elastic shell. This results in a kind of cushion structure. The moldings and/or semifinished products are introduced into the at least one secondary lumen through the lateral opening. The particles, moldings and/or semifinished products can be introduced manually, for example by means of a manually guided tool suitable for this purpose, semiautomatically or fully automatically. After it has been introduced, the thermoplastic elastomer material is expanded. To this end, the blowing agent present in the thermoplastic elastomer material is activated by action of heat or by action of energy in the broadest sense.

In one embodiment, the method comprises the step of: processing particles of the expandable thermoplastic elastomer material, there being formed a shaped cord pressed from the particles or a cushion structure having an elastic shell filled with the particles, and the expandable thermoplastic elastomer material in the form of the shaped cord or the cushion structure being introduced into the at least one secondary lumen through the lateral opening. The shaped cord can already be cut to a required or desired length before it is introduced into the at least one secondary lumen. Alternatively, the shaped cord is only cut to length after it has been introduced. To this end, one end of the shaped cord protruding from the lateral opening after introduction is removed. The elastic shell can be in the form of a bag or a mesh. In one embodiment, the elastic shell and the catheter tube are made of the same plastics material. This can achieve a particularly advantageous integral bond between the shell and the inner wall of the at least one secondary lumen. In a further embodiment, the elastic shell and the catheter tube are made of different materials.

In one embodiment, the expansion comprises: activating a blowing agent present in the expandable thermoplastic elastomer material, the blowing agent being activated by means of an energy input. The energy input can be effected by direct heating and/or irradiation of the catheter tube provided with the expandable thermoplastic elastomer material, for example via heating elements. Moreover, it is conceivable to heat over a naked flame. Alternatively, the heat can be input via a heat transfer medium, for example water vapor. Alternatively, the heat can be input via electromagnetic radiation to activate the blowing agent. Especially conceivable according to the inventors are irradiation with microwaves, infrared radiation and/or UV radiation and infrared laser beams and/or UV laser beams. In such an embodiment, it may be advantageous if the catheter tube and/or the thermoplastic elastomer material introduced into the at least one secondary lumen has an absorber for conversion of the radiation into heat. Also conceivable is heating by means of induction. In this case, for example, metallic particles can be embedded in the catheter tube and/or the thermoplastic elastomer material as absorbers. Moreover, the heat input softens the thermoplastic elastomer material and makes it flowable. The softened and flowable thermoplastic elastomer material is expanded by the activated blowing agent. The resulting internal forces bring about the formation of microscopic pores, which is manifested in macroscopic deformation and/or expansion of the thermoplastic elastomer material. After cooling and solidification, the thermoplastic elastomer material remains in its expanded state. In an expanded state, the thermoplastic elastomer material has a porous structure. A morphology of this porous structure (open-pore, closed-pore, pore size, degree of expansion) is controllable by different parameters. Parameters include, in particular, a proportional amount of the blowing agent used or present (“loading”), an intensity and duration of the energy input, a cooling rate and/or nucleation kinetics.

In one embodiment, the catheter tube is made of a meltable plastics material, and the at least one secondary lumen in the region of the distal tube end is sealed at least partially by means of a material plug integrally connected to the rest of the catheter tube, the material plug being formed by melted and resolidified plastics material of the distal tube end. The material plug forms a further seal of the at least one secondary lumen. Similar to the seal formed from the viscous sealing compound or the expanded thermoplastic elastomer material, the material plug counteracts unwanted collapse of the catheter tube in the radial direction. This achieves further improved properties of the catheter. The material plug consists of plastics material which was/has been melted earlier in the region of the distal tube end and was/has been introduced into the at least one secondary lumen, where it was/has been solidified. Accordingly, the material plug is integrally joined or bonded to the rest of the catheter tube and not, for instance, a component separate from the catheter tube. In other words: the material plug is an integral part and/or section of the catheter tube. Preferably, the molten plastics material is introduced through a distal opening of the at least one secondary lumen that is initially present on the catheter tube and/or a semifinished tube. Said distal opening is sealed by the formation of the material plug.

In one embodiment, a catheter tip is joined to the distal tube end, the catheter tip being integrally bonded to an end-face bonding surface of the catheter tube, and a distal end face of the material plug forming a section of the bonding surface. The end-face bonding surface of the catheter tube that is available for connection to the catheter tip is enlarged by the distal end-face of the material plug. In comparison to a solution without a material plug and/or with a mandrin, this can achieve increased strength of the integral bond. In this embodiment, the catheter tip is a component made separately from the catheter tube. The integral bond is preferably an adhesive bond, fusion bond and/or weld bond.

### BRIEF DESCRIPTION OF THE DRAWINGS

Further advantages and features of the disclosure will become clear from the following description of preferred exemplary embodiments of the disclosure, which are shown in the drawings.

FIG. 1 shows a schematic representation of a catheter according to the disclosure comprising a catheter tube,

FIG. 2 shows an enlarged longitudinal section of a distal end of the catheter as per FIG. 1, wherein a secondary lumen is sealed with a seal,

FIGS. 3 to 9 show schematically simplified representations to illustrate individual steps of different embodiments of methods according to the disclosure for producing the catheter as per FIGS. 1 and 2,

FIG. 10 shows an enlarged longitudinal section of a distal end of one embodiment of a catheter according to the disclosure, wherein the at least one secondary lumen is sealed distally by means of a material plug, and

FIGS. 11 to 14 show further schematic representations to illustrate individual method steps for producing the catheter as per FIG. 10, wherein the method steps are combinable with the method steps illustrated in FIGS. 3 to 9.

### DETAILED DESCRIPTION

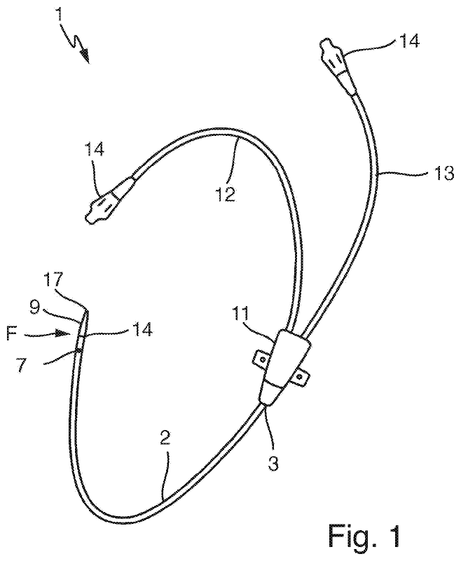

A catheter 1 as per FIG. 1 is intended for use in infusion therapy. In the present case, the catheter 1 is a two-lumen central venous catheter. Embodiments not shown in greater detail in a drawing are directed to three-lumen, four-lumen, five-lumen or multilumen catheters. The solution according to the disclosure is not limited to central venous catheters, but is also applicable to other medical catheters comprising a multilumen catheter tube.

The catheter 1 comprises a catheter tube 2.

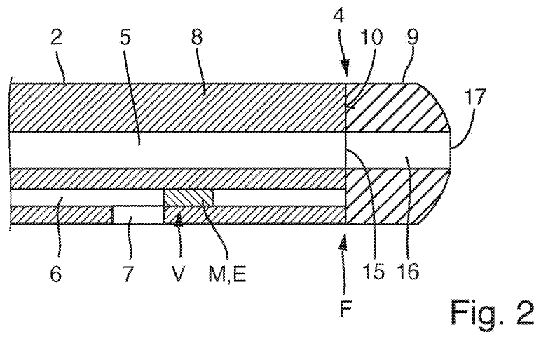

The catheter tube 2 is elongate between a proximal tube end 3 and a distal tube end 4 and has a main lumen 5 and at least one secondary lumen 6 (FIG. 2). The main lumen 5 and the at least one secondary lumen 6 are each elongate between the proximal tube end 3 and the distal tube end 4. The at least one secondary lumen 6 has a lateral opening 7. The lateral opening 7 is arranged between the proximal tube end 3 and the distal tube end 4 in the axial direction of the catheter tube 2. Moreover, the lateral opening 7 extends through a tube casing 8 of the catheter tube 2 in the radial direction of the catheter tube 2. The lateral opening 7 acts as an outlet opening and/or inlet opening, depending on the flow direction of a liquid to be administered or removed by means of the catheter 1.

The tube casing 8 can also be referred to as a tube wall or wall.

Moreover, the catheter 1 comprises a seal V (FIG. 2). The seal V is arranged in the at least one secondary lumen 6. At the same time, the seal V is introduced into the at least one secondary lumen 6 in the distal direction behind the lateral opening 7. In other words, the seal V is arranged between the lateral opening 7 and the distal tube end 4 in the axial direction of the catheter tube 2 and/or the at least one secondary lumen 6. In the embodiment shown, the seal V is introduced into the at least one secondary lumen 6 in the axial direction immediately behind the lateral opening 7 adjacent to said lateral opening 7.

According to the disclosure, the seal V can differ in design and/or nature:

In one embodiment, the seal V is formed from a sealing compound M introduced into the at least one secondary lumen 6 in a viscous state and cured within the at least one secondary lumen 6.

In a further embodiment, the seal V is formed from a thermoplastic elastomer material E introduced into the at least one secondary lumen 6 and expanded within the at least one secondary lumen 6.

FIG. 2 serves to illustrate both embodiments. In this respect, FIG. 2 is to be understood as schematically simplified.

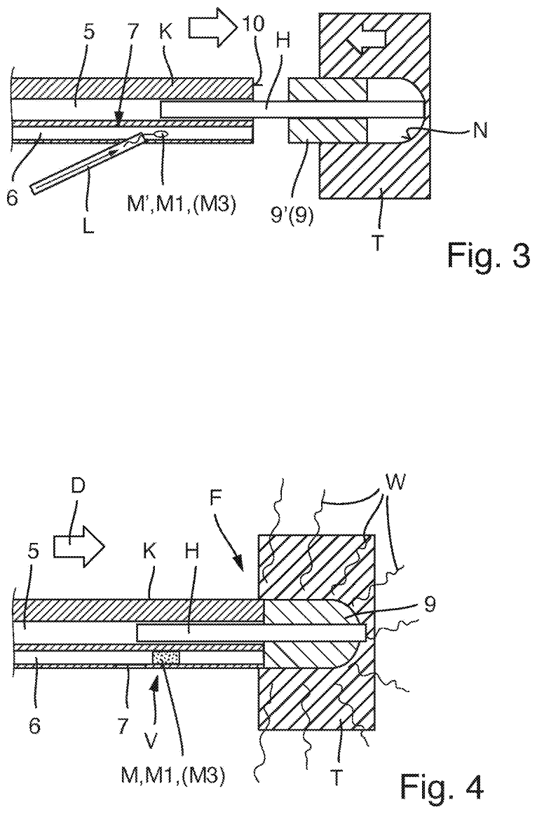

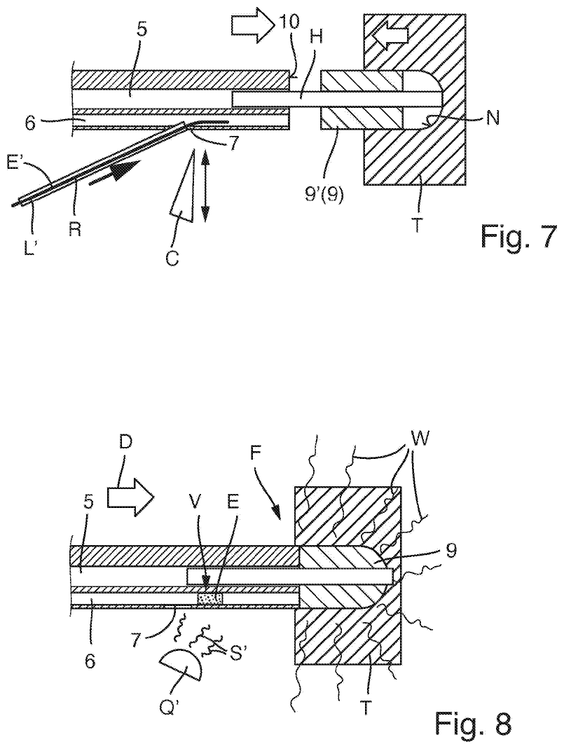

The embodiment with cured sealing compound M will be explained in greater detail below by means of FIGS. 3 to 6. The further embodiment with expanded thermoplastic elastomer material E will be described in detail by means of FIGS. 7 to 9.

Further features of the catheter 1 shown that are to be considered advantageous, but not essential in respect of the present disclosure, will be discussed first below.

In the present case, the catheter 1 has a catheter tip 9. The catheter tip 9 is joined to the distal tube end 4. In the present case, what is provided to this end is an integral bond or joint F between an end-face bonding surface 10 of the catheter tube 2 and a proximal end face of the catheter tip 9.

In an embodiment not shown in a drawing, the catheter 1 does not have a catheter tip. In a further embodiment not shown, the catheter tip is not in the form of a separately manufactured component subsequently joined to the distal tube end, but instead in the form of an integrally molded section of the distal tube end.

Apart from that, the catheter 1, aside from the seal V designed according to the disclosure, has a design and function known in principle to a person skilled in the art. Accordingly, the proximal tube end 3 is connected to a first supply line 12 and a second supply line 13 via a catheter hub 11. The two supply lines 12, 13 each have a fluid connector 14, which can be designed, for example, as a Luer connector or NRFit connector. In the present case, the first supply line 12 is assigned to the main lumen 5. In the present case, the second supply line 13 is assigned to the at least one secondary lumen 6. The two supply lines 12, 13 are fluidically connected to the respective lumen 5, 6 via the catheter hub 11 in a manner known in principle. Accordingly, the main lumen 5 has an inlet opening, not shown in greater detail, which is arranged in the region of the proximal tube end 3 and which is fluidically connected to the first supply line 12 via the catheter hub 11. The same applies, mutatis mutandis, to the at least one secondary lumen 6. The main lumen 5 has a distal outlet opening 15 in the region of the distal tube end 4. The distal outlet opening 15 opens into a proximal inlet opening of the catheter tip 9 that is not further identified (FIG. 2) and is connected to a distal outlet opening 17 of the catheter tip 9 via a lumen 16 of the catheter tip 9. The at least one secondary lumen 6 does not have such a distal opening. Present instead is the side opening 7. In the distal direction, the at least one secondary lumen 6 is sealed by means of the seal V.

The formation of the seal V from said sealing compound M is illustrated schematically by means of FIGS. 3 to 6. These figures relate to individual method steps of a method for producing the catheter 1 shown by means of FIGS. 1 and 2.

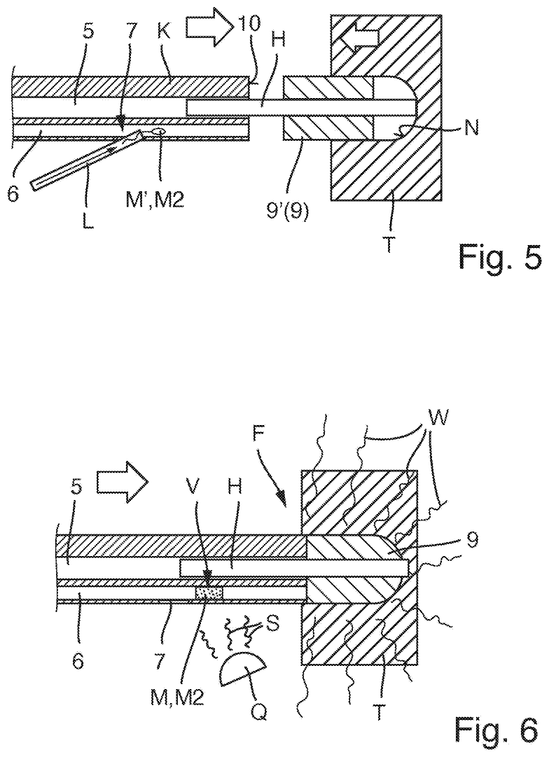

To form the seal V, the viscous sealing compound M′ is introduced into the at least one secondary lumen 6 (FIGS. 3 and 5). To this end, the viscous sealing compound M′ is introduced into the at least one secondary lumen 6 through the lateral opening 7. This is carried out in the present case using a tool L suitable for this purpose. By means of the tool L, the viscous sealing compound M′ can be injected, fed, filled and/or pressed into the at least one secondary lumen 6 in the distal direction through the lateral opening 7. In the embodiments presently shown, the introduction of the viscous sealing compound M′ is carried out together with a further step of the method for producing the catheter 1. This step will be explained in greater detail below by means of FIGS. 3 and 5 and is not absolutely necessary in respect of the present disclosure.

After introduction into the at least one secondary lumen 6, the (initially) viscous sealing compound M′ is cured. In this respect, the sealing compound following curing is called a cured sealing compound M or the cured sealing compound M. The viscous sealing compound M′ can be cured in different ways:

FIGS. 3 and 4 relate to one variant of the method in which the viscous sealing compound M′ cures under the influence of air humidity. FIGS. 5 and 6 relate to one variant of the method in which the viscous sealing compound M′ is cured under the action of a radiation S (FIG. 6). Accordingly, the two variants shown also differ in respect of the properties, composition and/or nature of the sealing compound M respectively used:

In the variant as per FIGS. 3 and 4, the sealing compound is and/or contains cyanoacrylate M1. The use of cyanoacrylate M1 allows in particular curing under the action of air humidity alone, and so a separate action of radiation (FIG. 6) can be dispensed with.

In a modification of the variant as per FIGS. 3 and 4, silicone M3 is used instead of the cyanoacrylate M1. This is illustrated by reference number M3 in parentheses in FIGS. 3 and 4.

In the variant as per FIGS. 5 and 6, the sealing compound M is and/or contains a polymer M2 crosslinkable and thus curable under the action of radiation S. The polymer M2 can be, for example, a resin, a monopolymer and/or a prepolymer.

The radiation S can be heat radiation or UV light, depending on the composition, properties and/or nature of the polymer M2 used. The radiation S is emitted by a radiation source Q configured for this purpose.

As already mentioned, further steps for producing the catheter 1 are illustrated by means of FIGS. 3 and 4. These steps relate to the integral joining of the catheter tip 9 to the distal tube end 4. For this purpose, the catheter tip 9 is accommodated on a mold T and is threaded onto a holding needle H to this end. One end of the holding needle H is fixed to the mold T. In the configuration shown by means of FIG. 3, the catheter tip 9 does not yet have its final contour, and so the catheter tip 9 can also be called a semifinished product 9′. The mold T has a negative shape N complementary to the end-face contour of the catheter tip 9 to be produced. The catheter tube 2 is also threaded, with its distal end 4 first, onto the holding needle H. To this end, the holding needle H is inserted into the main lumen 5 in the proximal direction. The catheter tube 2, the catheter tip 9 or semifinished product 9′ and the mold T are brought together in the axial direction. This is illustrated by means of the opposite arrows drawn in schematically in FIG. 3.

The mold T is heated. The heated mold T causes action of heat W on the catheter tip 9 and/or the semifinished product 9′ and the distal tube end 4. Moreover, an axial compressive force D is applied, by means of which the distal tube end 4 together with the catheter tip 9 is pressed into the mold T, more precisely the negative shape N thereof. Firstly, the distal end contour of the catheter tip 9 is formed by the action of heat and compressive force. In addition, the catheter tip 9 is integrally bonded to the end-face bonding surface 10 of the distal tube end 4. The integral bond is achieved by melting, cooling and solidification of the material used for the catheter tube 2 and/or the catheter tip 9.

In the present case, the material used is a meltable plastics material K. The catheter tip 9 can be made of precisely this meltable plastics material K or made of a different (meltable) plastics material, for example one having lower hardness.

In the variant of the method as per FIGS. 5 and 6, the catheter tip 9 is joined in a corresponding manner, and so, to avoid repetition, reference is made to what has already been stated in relation to FIGS. 3 and 4.

FIGS. 7 to 9 relate to the embodiment in which the seal V is formed from the expanded thermoplastic elastomer material E. In an as yet unexpanded state or in an incompletely expanded state, the thermoplastic elastomer material is identified by the reference sign E′. In the present case, the expandable thermoplastic elastomer material is EP-TPE. To the knowledge of the inventors, EP-TPE is in any case not a common material in the field of medical technology.

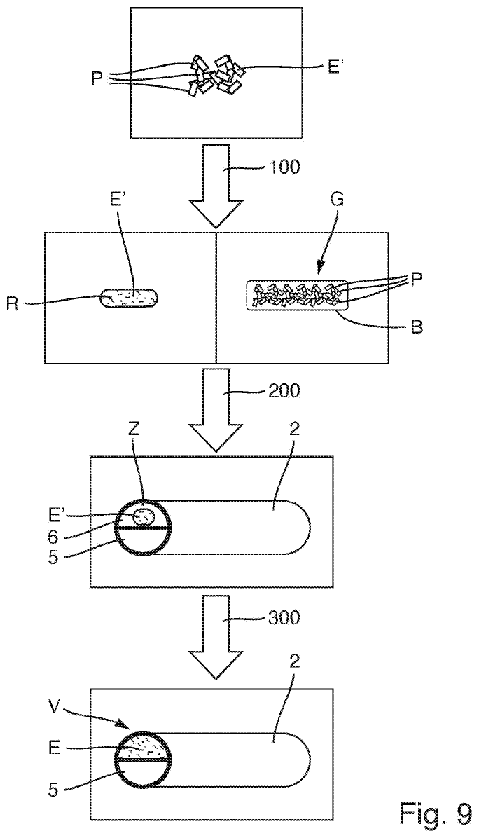

The basis for the presently discussed method variants are particles of the expandable thermoplastic elastomer material E′, which are depicted schematically in greatly simplified form by means of FIG. 9. The elastomer material E′ and/or particles P comprise a blowing agent that is not further identified. In any case, this is familiar to a person skilled in the art in the field of plastics technology. Said blowing agent is activatable in a manner that will be described in greater detail, the activation of the blowing agent bringing about said expansion.

The thermoplastic elastomer material E′ can be introduced into the at least one secondary lumen 6 through the lateral opening 7 in different ways.

In one variant of the method, the particles P are filled into the at least one secondary lumen 6 through the lateral opening 7. Thereafter, the blowing agent is activated, the particles P are expanded, and the seal V is formed as a result. The introduction of the particles P and the activation of expansion are illustrated schematically in FIG. 9 by means of arrows 200, 300.

In other variants of the method, processing 100 of the particles P is carried out first before introduction 200. In this case, the particles P are processed into moldings, semifinished products or the like that differ in design and/or nature.

In one variant, the particles P are pressed into a shaped cord R. The shaped cord R is depicted schematically in simplified form in FIG. 9. As shown in FIG. 7, the shaped cord R is introduced into the at least one secondary lumen 6 through the lateral opening 7 by means of a tool L′ suitable for this purpose. After it has been introduced, the shaped cord R is shortened to a required and/or desired length in the region of the lateral opening 7 by means of a cutting tool C. Both the introduction by means of the tool L′ and the separation by means of the cutting tool C can be carried out manually, semiautomatically, or fully automatically. Moreover, it is possible that the shaped cord R is already shortened to the required and/or desired length before it is introduced. A cross-section of the shaped cord R that is not further identified is smaller than a cross-section of the at least one secondary lumen 6. As a result, the—as yet unexpanded—shaped cord R can be readily advanced within the lumen 6 in the axial direction. In other words, a radial gap Z is initially present between the shaped cord R and an inner wall of the at least one secondary lumen 6 that is not further identified. The radial gap Z is illustrated schematically in FIG. 9 and is completely sealed after activation 300, i.e., after the expansion of the thermoplastic elastomer material.

In another variant, the particles P are filled into an elastic shell B before introduction 200. This forms a cushion structure G. The cushion structure G is depicted schematically in simplified form by means of FIG. 9. The cushion structure G can be pushed into the at least one secondary lumen 6 through the lateral opening 7, similar to the shaping cord R. This is followed by activation and/or expansion 300.

Irrespective of whether the expandable elastomer material E is introduced into the at least one secondary lumen 6 in the form of the particles P, the shaped cord R or the cushion structure G, the subsequent expansion to form the seal V is carried out with the input of an energy S′ (FIG. 8). The energy S′ is generated by means of an energy source Q′ configured for this purpose and is input into the catheter tube 2 provided with the elastomer material E′. The energy S′ is input in different ways in different variants of the method. In one variant, the energy S′ is input as heat. In this case, the energy source Q′ is a heating element. Moreover, it is conceivable to heat over a naked flame. Moreover, a heat transfer medium, for example water vapor, can be used. In another variant, the energy S′ is input as electromagnetic radiation. For example, the electromagnetic radiation can be input as microwave radiation, infrared radiation and/or UV radiation. In order to allow an appropriate conversion of the input electromagnetic radiation into heat, it may be necessary in variants of the method to add additives suitable for this purpose to the thermoplastic elastomer material. Moreover, the energy S′ can be input by induction. In this case, a metallic component, for example in the form of metal particles, can be added to the thermoplastic elastomer material.

Apart from that, FIGS. 7 and 8 also illustrate the already discussed joining of the catheter tip 9. To avoid repetition, reference is made to what has been stated above.

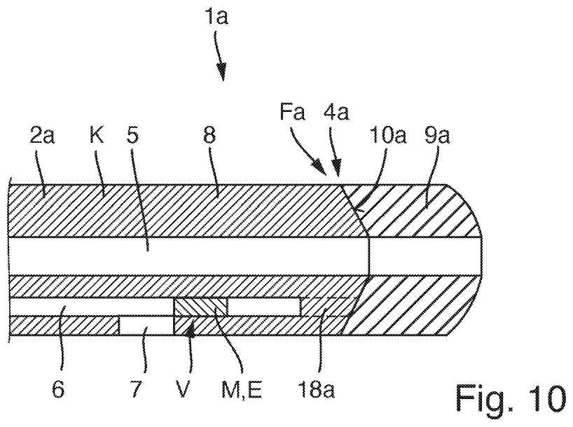

FIG. 10 shows a further embodiment of a catheter 1a. The catheter 1a is substantially identical to the catheter 1 explained above. Accordingly, the catheter 1a likewise has within the at least one secondary lumen 6 a seal V formed from cured sealing compound M or expanded thermoplastic elastomer material E. The seal V is also formed in the catheter 1a in the manner explained above and/or using one of the methods explained above. To avoid repetition, only essential differences between the catheter 1a and the catheter 1 will be explained below.

The essential difference is that the catheter 1a has a material plug 18a in the region of the distal tube end 4a. The material plug 18a is assigned to the at least one secondary lumen 6. The material plug 18a is integrally joined to the rest of the catheter tube 2a, which is illustrated in FIG. 10 by means of the two dashed lines. In other words, the material plug 18a is an integral part and/or section of the catheter tube 2a. The material plug 18a acts as an additional or further seal. In this respect, the material plug 18a seals the at least one secondary lumen 6 at the end face in the region of the distal tube end 4a. Moreover, the material plug 18a brings about mechanical stabilization of the distal tube end 4a, in particular in the radial direction.

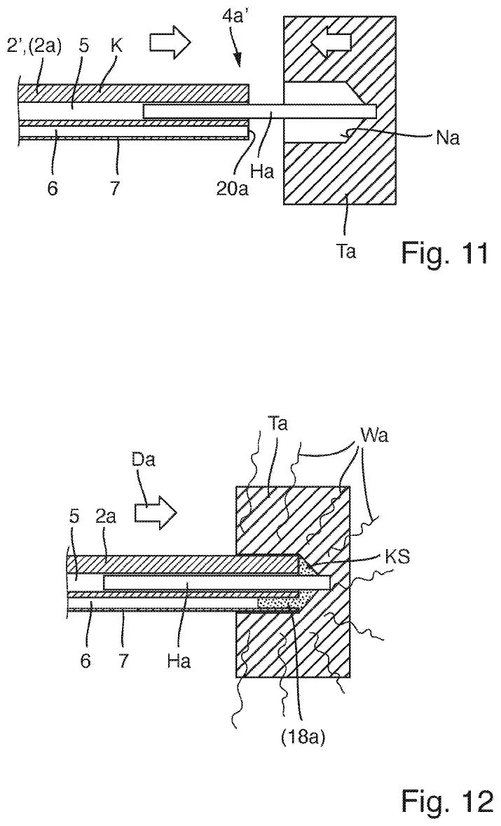

The material plug 18a is formed by melted and resolidified plastics material K of the distal tube end 4a. In the present case, the melting and resolidification is carried out on the basis of a semifinished product 2a′ for producing the catheter tube 2a that is shown by means of FIG. 11 and that can also be referred to as a semifinished tube. This will be described in detail below. In simple terms, the meltable plastics material K is melted in the region of the distal tube end of the semifinished product 2a′ under the action of heat, introduced into the at least one secondary lumen 6 of the semifinished product 2a′ in the proximal direction, and then solidified to form the material plug 18a.

In a completely formed state (FIG. 10), the material plug 18a seals the at least one secondary lumen 6, more precisely the cross-section thereof, in the radial direction without any gaps. This ensures, firstly, complete sealing of the at least one secondary lumen 6 in the region of the distal tube end 4a. Moreover, the already mentioned improved mechanical stability of the distal tube end 4a can be achieved in the radial direction. This is in contrast to solutions known from the prior art, in which the at least one secondary lumen is sealed in the region of the distal tube end by means of a mandrin which has naturally been pushed into the at least one secondary lumen with radial clearance.

Individual steps of a method for producing the catheter 1a will be explained below by means of FIGS. 11 to 14.

The method illustrated in the present case is based on the semifinished product 2a′ shown in FIG. 11. To form the material plug 18a, the meltable plastics material K is first melted in the region of the distal tube end 4a′. To this end, in the present case, the semifinished product 2a′, more precisely the distal tube end 4a′ thereof, is threaded onto a holding needle Ha (FIG. 11). To this end, the holding needle Ha is inserted into the main lumen 5 of the semifinished product 2a′ in the proximal direction. One end of the holding needle Ha is attached to a mold Ta. The mold Ta is heated to melt the plastics material K. The semifinished product 2a′ and the mold Ta are brought together in the axial direction. This is illustrated by means of the oppositely oriented arrows shown in FIG. 11. Here, the semifinished product 2a′ is guided in the radial direction by means of the holding needle Ha. The heated mold Ta causes an action of heat Wa (FIG. 12). Under the action of heat Wa, the plastics material K melts in the region of the distal tube end 4a′. At the same time, an axial compressive force Da is applied to the semifinished product 2a′ and/or the catheter tube 2a (FIG. 12). Under the action of the axial compressive force Da and/or the mold Ta, the molten plastics material is pressed into the at least one secondary lumen 6 in the proximal direction through a distal opening 20a (FIG. 11) of the at least one secondary lumen 6 that is initially present on the semifinished product 2a′. The molten plastics material is identified by reference sign KS in FIG. 12 and can also be referred to as a plastics melt or melt cushion.

Thereafter, the catheter tube 2a is removed from the mold Ta. The plastics material introduced into the at least one secondary lumen 6 in the manner described above solidifies and thereby forms said material plug 18a. In the configuration shown by means of FIG. 12, the plastics melt KS is not solidified or in any case not yet completely solidified in the region of the at least one secondary lumen 6, and so the material plug 18a is not yet formed or in any case not completely formed. Accordingly, reference sign 18a is in parentheses in FIG. 12.

In the embodiment shown, the mold Ta has a negative shape Na complementary to the shaping of the end-face bonding surface 10a of the catheter tube 2a. The end-face bonding surface 10a is beveled on the basis of the end-face shaping of the semifinished product 2a′ (FIG. 11). This results in a larger effective contact area for forming the integral bond Fa (FIG. 14). In this respect, reference can also be made to beveling of the distal tube end or to a beveled distal tube end 4a.

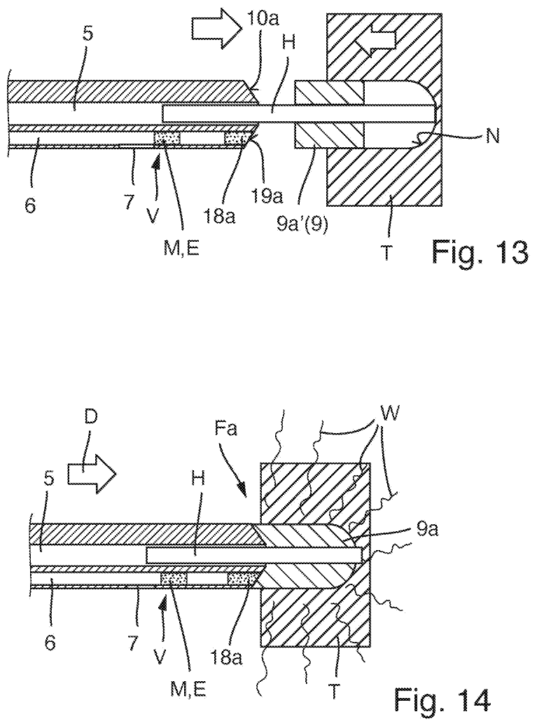

After beveling of the distal tube end 4a, the catheter tip 9a is integrally joined to the bonding surface 10a (FIGS. 13 and 14). To this end, the catheter tip 9a is again accommodated on the already discussed mold T and threaded onto the holding needle H. In the configuration shown by means of FIG. 13, the catheter tip 9a does not yet have its final contour, and so it can again be called a semifinished product or semifinished tip 9a′. The formation of the integral bond Fa between the catheter tube 2a and the catheter tip 9a is in principle carried out in the manner already explained by means of FIGS. 3 and 4 and FIGS. 5 and 6. In this respect, to avoid repetition, reference is made to what has already been stated.

In contrast to the integral bond F in the case of the catheter 1, the integral bond Fa in the case of the catheter 1a is additionally formed between the material plug 18a and the catheter tip 9a. The material plug 18a therefore effectively brings about a further enlargement of the effective bonding surface. In other words, a distal end face 19a of the material plug 18a forms a section of the bonding surface 10a.

What is claimed:

1. A catheter comprising:
a catheter tube; and
a seal,
the catheter tube comprising a main lumen and at least one secondary lumen,
the main lumen and the at least one secondary lumen each being elongate between a proximal tube end and a distal tube end,
the at least one secondary lumen having a lateral opening arranged between the proximal tube end and the distal tube end in an axial direction of the catheter tube,
the lateral opening extending through a tube casing of the catheter tube in a radial direction of the catheter tube,
the seal being introduced into the at least one secondary lumen distally behind the lateral opening and sealing the at least one secondary lumen at least partially,
the seal being formed from a sealing compound introduced into the at least one secondary lumen in a viscous state and cured within the at least one secondary lumen.

2. The catheter according to claim 1, wherein the sealing compound is and/or contains cyanoacrylate.

3. The catheter according to claim 1, wherein the sealing compound is and/or contains silicone.

4. The catheter according to claim 1, wherein the sealing compound is and/or contains a crosslinkable polymer.

5. The catheter according to claim 1, wherein the catheter tube is made of a meltable plastics material, and wherein the at least one secondary lumen in a region of the distal tube end is sealed at least partially by a material plug that is integrally connected to the catheter tube, the material plug being formed by melted and resolidified plastics material of the distal tube end.

6. The catheter according to claim 5, wherein a catheter tip is joined to the distal tube end, the catheter tip being bonded to an end-face bonding surface of the catheter tube, and a distal end face of the material plug forming a section of the bonding surface.

7. The catheter according to claim 1, wherein the seal is formed from a thermoplastic elastomer material that is expanded within the at least one secondary lumen.

8. The catheter according to claim 7, wherein the catheter tube is made of a meltable plastics material, and wherein the at least one secondary lumen in a region of the distal tube end is sealed at least partially by a material plug that is integrally connected to the catheter tube, the material plug being formed by melted and resolidified plastics material of the distal tube end.

9. The catheter according to claim 8, wherein a catheter tip is joined to the distal tube end, the catheter tip being bonded to an end-face bonding surface of the catheter tube, and a distal end face of the material plug forming a section of the bonding surface.

10. A method for producing the catheter according to claim 1, the method comprising the steps of:
introducing the sealing compound into the at least one secondary lumen through the lateral opening; and
curing the sealing compound to form the seal.

11. A method for producing the catheter according to claim 7, the method comprising the steps of:
introducing the thermoplastic elastomer material into the at least one secondary lumen through the lateral opening; and
expanding the thermoplastic elastomer material to form the seal.

12. The method according to claim 11, further comprising the step of:
processing particles of the thermoplastic elastomer material to form a shaped cord pressed from the particles or a cushion structure having an elastic shell filled with the particles,
the shaped cord or the cushion structure being introduced into the at least one secondary lumen through the lateral opening.

13. The method according to claim 11, wherein the step of expanding the thermoplastic elastomer material to form the seal comprises activating a blowing agent present in the expandable elastomer material, the blowing agent being activated by an energy input.
